(19) United States
(12) Patent Application Publication
(10) Pub. No.: US 2007/0225734 A1
(43) Pub. Date: Sep. 27, 2007

(54) Systems and methods for less invasive resolution of maladies of tissue including the appendix, gall bladder, and hemorrhoids

(75) Inventors: Stephen Graham Bell, Roma (IT); Wayne A. Noda, Mission Viejo, CA (US)

Correspondence Address: ROGITZ & ASSOCIATES, 750 B STREET, SUITE 3120, SAN DIEGO, CA, 92101, US

(73) Assignee: MINOS MEDICAL

(21) Appl. No.: 11/601,199

(22) Filed: Nov. 17, 2006

Related U.S. Application Data
(60) Provisional application No. 60/784,694, filed on Mar. 22, 2006.

(51) Int. Cl. A61B 17/10

(52) U.S. Cl. 606/139

(57) ABSTRACT

Systems and methods for transanal inversion and removal of the gall bladder, appendix, and hemorrhoids.

### PRIORITY CLAIM

This application claims priority from U.S. provisional application No. 60/784,694, filed Mar. 22, 2006.

### FIELD OF THE INVENTION

The present invention relates to less invasive surgical procedures, and more particularly to procedures that require no incision into the human body.

### BACKGROUND OF THE INVENTION

Less invasive procedures have been developed to resolve, e.g., by removal, maladies of tissue. An example of such a procedure is laparoscopy, in which a small incision is made near the navel and a device known as a laparoscope is inserted through the incision to view and/or remove tissue in the abdomen.

As understood herein, current less invasive procedures, while avoiding large incisions, nonetheless require incisions be made into the body through the abdominal wall, and any incision carries some degree of risk and patient discomfort. As further recognized herein, some commonly encountered maladies, including appendicitis, hemorrhoids, and gall bladder derangements, can be surgically addressed without making any incision at all, but rather by advancing surgical instruments through a natural body orifice such as the anus. The present invention still further recognizes, however, that aspects of such a procedure raise additional considerations that must also be addressed.

### SUMMARY OF THE INVENTION

A tissue inversion catheter assembly includes a flexible elongated catheter body that is transanally advanceable into a patient. An elongated inverter is advanceable distally through the catheter body into a patient tissue sought to be inverted, e.g., into the appendix or gall bladder or other tissue. The inverter includes structure for urging the tissue against the inverter so that upon proximal retraction of the inverter the tissue inverts upon itself.

The structure for urging the tissue against the inverter may include plural vacuum holes formed in the inverter and communicating, via a vacuum lumen of the inverter, with a source of vacuum external to the patient. The inverter can define an external surface formed with spiral or ringed ridges, and the vacuum holes may be formed closer to proximal sides of the ridges than to distal sides. If desired, the inverter can reciprocate within a vacuum sealing sleeve in the catheter assembly, with the sleeve being positionable at the entry of the tissue being inverted to hold vacuum in the tissue.

Alternatively, the structure for urging the tissue against the inverter may include a gripping element that is manipulable between a wide configuration, wherein the gripping element is positioned near the tissue, and a gripping configuration, wherein the gripping element grasps the tissue between gripping arms.

In some embodiments a ligator holder can be slidably disposed in the inversion catheter assembly and can have a distal end bearing a ligation element. The ligator holder may be slidable past the tissue to a ligation point to position the ligation element around the tissue. A tightener such as a ligation loop can be coupled to the ligator holder to cinch the ligation element around the tissue. If desired, an excision tube bearing a snare that can be advanced distally away from the excision tube to snare the tissue after ligation for removing the tissue from the patient.

In non-limiting embodiments the catheter body is coupled to an endoscope to enable a person to view tissue as the catheter body is advanced into the patient. The inversion catheter assembly can if desired include structure for facilitating ultrasonic and/or fluoroscopic guiding at least of the inverter.

As set forth further below, in some implementations a manipulator extends through the endoscope and is advanceable into the tissue. The manipulator is coupled to an external manipulation control for moving the manipulator within the tissue to determine whether the tissue has undesirable adhesions to nearby tissue prior to attempting inversion of the tissue. In one embodiment, the manipulator includes a stylet wire that is sufficiently rigid to straighten the tissue. In another embodiment, the manipulator includes a manipulator catheter having an actuator wire anchored near a distal end thereof, with the manipulator catheter being sufficiently flexible to adhere to the contour of the tissue. The actuator wire can be coupled to an actuator external to the patient and manipulable to cause the manipulator catheter to bend within the tissue, causing the tissue to thereby bend.

The ligation element may include a loop having a textured inner surface to facilitate gripping tissue. The loop can be formed by drawing an elongated flexible ligation member through a locking eye. In non-limiting embodiments the ligation member has a textured surface to facilitate frictional self-locking between the locking eye and the ligation member. In other embodiments the ligation element includes a loop and a tightening member extending away from the loop a direction that is coaxial to the loop to tighten the loop around tissue.

A guidewire may be provided to extend through the catheter body. The guidewire may have an inflatable balloon at a distal end thereof to hold the guidewire within an appendix. Or, the guidewire may have expandable wings at a distal end thereof to hold the guidewire within an appendix.

In another aspect, a method for appendectomy in a patient includes advancing an inverter into the appendix of the patient, and using the inverter to invert the appendix. The method also includes removing the inverted appendix from the patient.

In another aspect, a method for appendectomy in a patient includes transanally advancing a catheter toward the appendix of the patient, and using the catheter to remove the appendix from the patient.

In still another aspect, a manipulator for moving an appendix include a manipulator catheter advanceable into the appendix. The manipulator catheter is coupled to an external manipulation control for moving a manipulator element inside the appendix to determine whether the appendix has restrictive anatomy or undesirable adhesions to nearby tissue.

In yet another aspect, a method for determining whether an appendix is subject to undesirable adhesions to tissue nearby the appendix includes advancing a manipulator into the appendix, and moving the manipulator to thereby move the appendix. Using fluoroscopy, it is determined whether the appendix has adhesions to nearby tissue.

In another aspect, a ligation element includes a loop having a textured inner surface and/or ribs to facilitate gripping tissue.

In another aspect, a ligation element includes a loop and a tightening member extending away from the loop in a direction perpendicular to the plane of the loop to tighten the loop around tissue.

In another aspect, a method for removing a gall bladder from a patient includes advancing an inverter into the gall bladder and using the inverter to invert the gall bladder. The method also includes removing the inverted gall bladder from the patient.

In still another aspect, a method for gall bladder removal in a patient includes transanally advancing a catheter toward the gall bladder of the patient, and using the catheter to remove the gall bladder from the patient.

The details of the present invention, both as to its structure and operation, can best be understood in reference to the accompanying drawings, in which like reference numerals refer to like parts, and in which:

### BRIEF DESCRIPTION OF THE DRAWINGS

FIG. 1 is a perspective view of the appendix manipulator being advanced into the appendix;

FIG. 2 is a perspective view of the appendix manipulator positioned in the appendix;

FIGS. 3 and 4 are perspective views of a first embodiment of the appendix manipulator manipulating the appendix to identify adhesions;

FIGS. 5 and 6 are perspective views of a second embodiment of the appendix manipulator manipulating the appendix to identify adhesions;

FIG. 7 is a cross-sectional view as seen along the line 7-7 in FIG. 6;

FIG. 8 is a perspective view of the proximal control portion of the manipulator device;

FIG. 9 is a perspective view of the manipulator combined with a tissue removal catheter, with portions of the overtube cut away for clarity;

FIG. 10 is a perspective view of the proximal control portion of the catheter shown in FIG. 9;

FIG. 10A is a perspective view of an alternate proximal control portion of the catheter shown in FIG. 9;

FIG. 11 is a side view of the inversion and excision elements of the catheter shown in FIG. 9 positioned adjacent the appendix;

FIG. 12 is a side view of the inversion and excision elements with the appendix inverted;

FIG. 13 is a side view of the inversion and excision elements with the appendix inverted and the ligating loop closed;

FIG. 14 is a side view of the inversion and excision elements with the appendix inverted, the ligating loop closed, and the cautery snare advanced onto the appendix;

FIG. 15 is a side view showing the excision element trimming the ligating loop in phantom and in solid snaring and retrieving the appendix;

FIG. 15A is a side view showing an alternate trimming mechanism;

FIGS. 16-20 are additional side views showing an alternate inverting catheter in combination with an endoscope and vacuum sealing sleeve for removing the appendix, with the sleeve shown in phantom in FIG. 16;

FIG. 21 is a perspective view of the distal portion of the catheter shown in FIGS. 16-20;

FIG. 22 is a perspective view of the proximal control portion of the catheter shown in FIGS. 16-21;

FIG. 23 is a perspective view of the inverter showing the vacuum holes;

FIG. 23A is a perspective view of an alternate inverter;

FIG. 24 is an enlarged view of a portion of the catheter shown in FIG. 23, illustrating a preferred location of vacuum holes;

FIGS. 25-30 are side views showing an alternate inversion catheter that grips tissue such as a hemorrhoid or the appendix and that is guided under fluoroscopy or ultrasound without the need for an endoscope;

FIGS. 31-33 are side views of a tissue ligating device;

FIGS. 34 and 35 show a ligating tie;

FIGS. 36 and 37 show an alternate tie;

FIGS. 38 and 39 are side views of a balloon-anchored guidewire; and

FIG. 40 is a side view of a wing-anchored guidewire.

### DETAILED DESCRIPTION OF THE PREFERRED EMBODIMENT

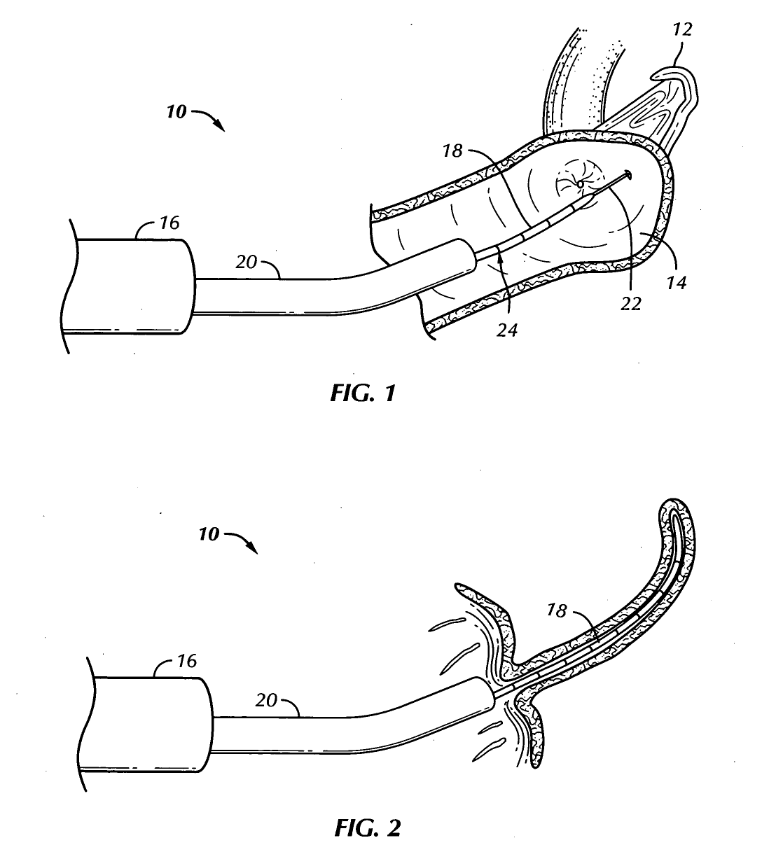

Referring initially to FIGS. 1 and 2, a catheter assembly is shown, generally designated 10, for manipulating and/or inverting an appendix 12 of a patient into the patient's cecum 14 to perform an appendectomy. It is to be understood that while the appendix is discussed for disclosure purposes, the catheter assembly 10 may also be used to invert the gall bladder for removal and to remove other tissue, e.g., to remove hemorrhoids. In any case, the catheter assembly 10 is advanced into the patient transanally, i.e., through the anus, so that no incision need be made to remove the appendix.

In the non-limiting implementation shown in FIGS. 1 and 2, the catheter assembly 10 includes a hollow overtube 16 through which an elongated flexible catheter-like manipulator 18 can be advanced. The manipulator 18 can be advanced through a lumen of an endoscope 20, described further below, which itself can be disposed in the overtube 16. In other embodiments no endoscope need be provided, with catheter guidance being effected by ultrasound or fluoroscopy as set forth further below. Even when an endoscope is provided, manipulation of the appendix can be visualized using ultrasound or fluoroscopy or MRI or CAT scan.

As shown in FIG. 1, a guidewire 22 can be disposed in a lumen of the manipulator 18 and the manipulator 18 slid over the guidewire 22. The guidewire 22 can be guided into the appendix by a surgeon viewing the appendix using the endoscope 20, and as disclosed further below may be anchored inside the appendix. Once the guidewire 22 is positioned in the appendix, the manipulator 18 can be advanced into the appendix 12 as shown in FIG. 2 and if desired radiopaque fluid infused into the appendix through the manipulator 18. In addition or in lieu of such infusion, the manipulator 18 can include axially-spaced radiopaque bands 24 that can be regarded as depth markings, for viewing of the manipulator 18 within the appendix 12 using fluoroscopy principles known in the art. Alternatively, ultrasonic imaging may be used.

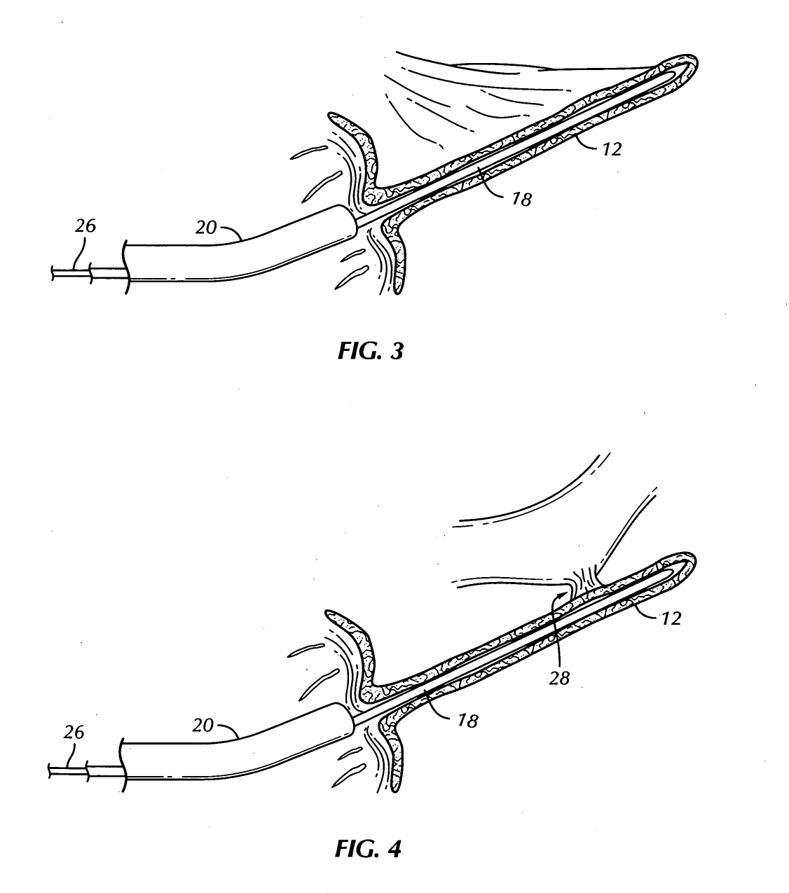

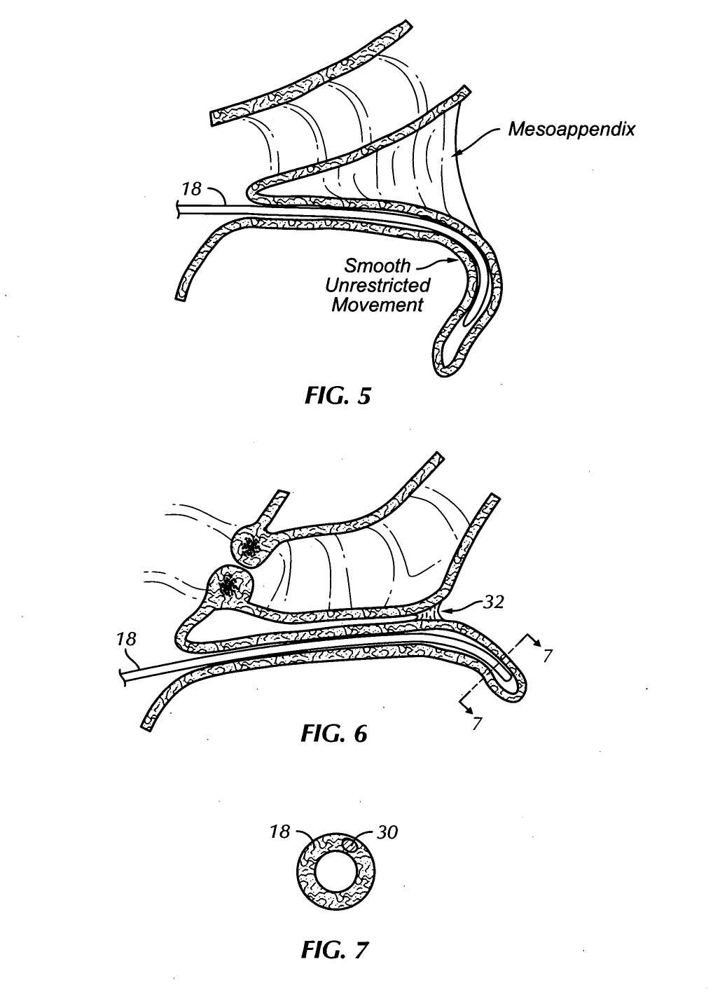

FIGS. 3 and 4 show a first alternate structure for manipulating the appendix. As shown, a relatively stiff stylet wire 26 can be advanced through the manipulator 18 to straighten the appendix 12. The stylet wire 26 preferably has an atraumatic tip to prevent perforation of the appendix. The stylet wire 26, which may be made of nitonol, possesses sufficient stiffness to straighten the appendix 12, and as the appendix 12 is straightened, any abnormal engagement 28 (collectively referred to herein as “adhesions”) of the appendix 12 with nearby tissue will appear under fluoroscopy (or ultrasonic imaging) as “tenting.”Alternatively, as shown in FIGS. 5-7 an actuator wire 30 can be embedded in the wall of the manipulator 18 and anchored near a distal end thereof. In this embodiment, the manipulator 18 is sufficiently flexible to adhere to the contour of the appendix 12 as shown in FIG. 5. The actuator wire 30 is coupled to an actuator (described further below) that is external to the patient and that is manipulable to cause the manipulator 18 to bend within the appendix 12, causing the appendix to thereby bend or turn. Abnormal adhesions (as at 32 in FIG. 6) can be visualized under fluoroscopy/ultrasonic imaging.

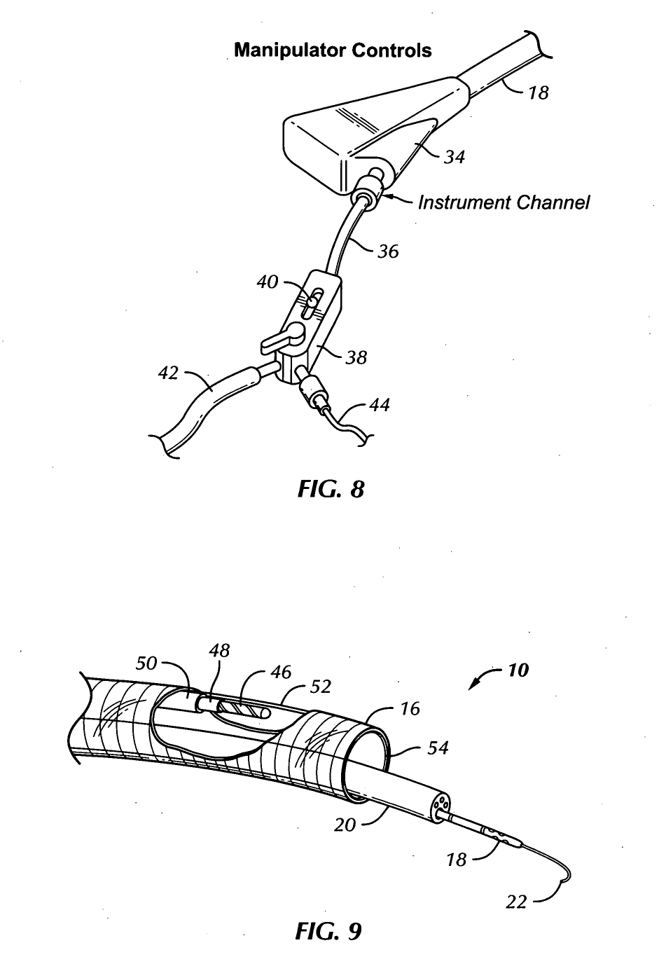

FIG. 8 shows non-limiting external controls for operating the manipulator 18. A hub 34 is provided (potentially as part of the endoscope 20) from which the manipulator 18 extends. An instrument channel 36 extends proximally away from the hub 34 and terminates in a tip control handle 38. A manipulable tip control element 40 such as a lever can be movably engaged with the handle 38 and coupled to, e.g., the actuator wire 30 shown in FIG. 7 so that when the element 40 is moved, the wire 30 is tensioned, bending the manipulator 18 at the distal end. The manipulator 18 may have multiple lumens including a suction lumen that communicates with a vacuum device through a suction tube 42 and an irrigation lumen that communicates with a source of irrigating fluid/fluoroscopy fluid through an irrigation tube 44, both of which tubes 42, 44 are connected to the handle 38 as shown.

FIG. 9 shows additional details of the catheter assembly 10 shown in FIG. 1. In addition to the endoscope 20 with manipulator 18, a hollow flexible inverter catheter 46 may extend through the overtube 16 as shown. The inverter catheter 46 can be slidably disposed in a hollow flexible sealing sleeve 48 which in turn may be slidably disposed in a hollow inverter outer cannula 50 within the overtube 16, for purposes to be shortly disclosed.

As also shown in FIG. 9, the overtube 16 may slidably bear an elongated hollow flexible ligator catheter 52 through which a ligating cord can extend to terminate in a loop 54 for suturing purposes to be shortly disclosed. The loop 54 can circumscribe the distal inner periphery of the overtube 16 as shown.

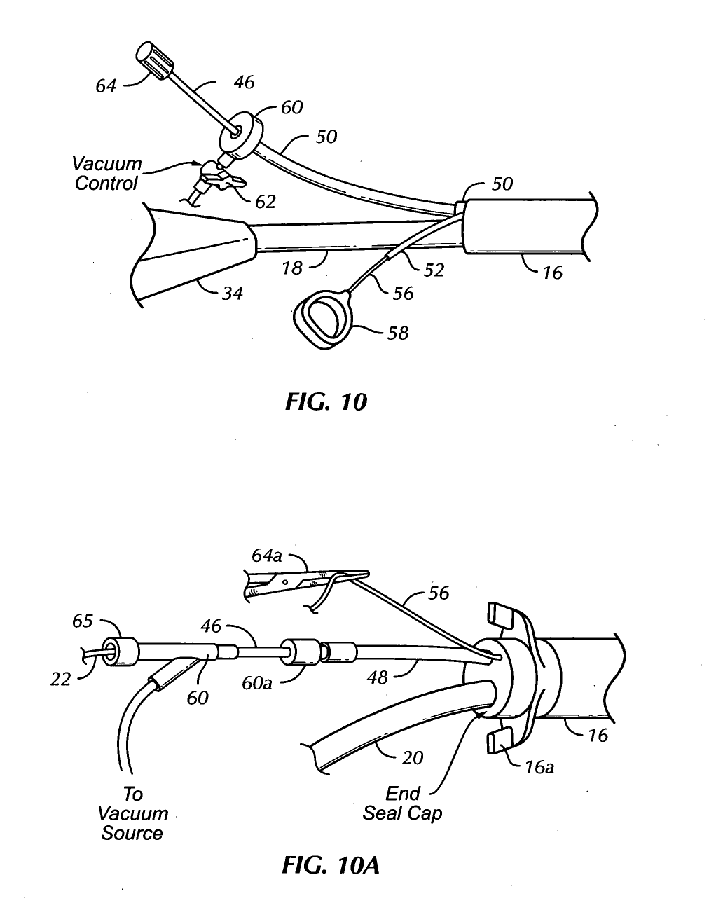

FIG. 10 shows non-limiting proximal surgical controls for operating the elements discussed above. The ligator catheter 52 extends proximally beyond the overtube 16 outside the patient, and the ligating cord 56 extends through the ligator catheter 52 and terminates in a loop tensioner handle 58. It will readily be appreciated that a surgeon can pull the handle 58 distally to tighten the loop 54 shown in FIG. 9.

Also, the inverter sealing sleeve 48 extends proximally beyond the overtube 16, with the inverter catheter 46 extending beyond the sleeve 48 through a vacuum hub 60 and with the sealing sleeve 48 being coupled to the hub 60. The vacuum hub 60 is connected to a vacuum control knob 62 and to a source of vacuum for selectively evacuating a vacuum lumen in the inverter catheter 46 in accordance with disclosure below by appropriately moving the control knob 62. The inverter catheter 46 terminates in a manipulable inverter control handle 64 for purposes to be shortly disclosed.

FIG. 10A shows alternate structure to that shown in FIG. 10. The seal sleeve 48 of the ligator catheter 52 extends proximally beyond the overtube 16 outside the patient, and the ligating cord 56 extends out of the overtube 16 for manipulation by a gripping device 64A such as pliers. As shown, the sealing sleeve 48 terminates in a Tuohy-Borst valve 60A, with the inverter catheter 46 extending through the valve 60A and terminating in a vacuum hub 60. The vacuum hub 60 is connected to a source of vacuum for selectively evacuating a vacuum lumen in the inverter catheter 46 in accordance with disclosure below. The hub 60 can be manipulated as appropriate to move the inverter catheter 46. The guidewire 22 extends through a guidewire seal 65 of the hub 60 in accordance with guidewire sealing means known in the art. The entire overtube 16 can be moved by grasping a torque handle 16A of the overtube 16 and moving the overtube 16 as desired.

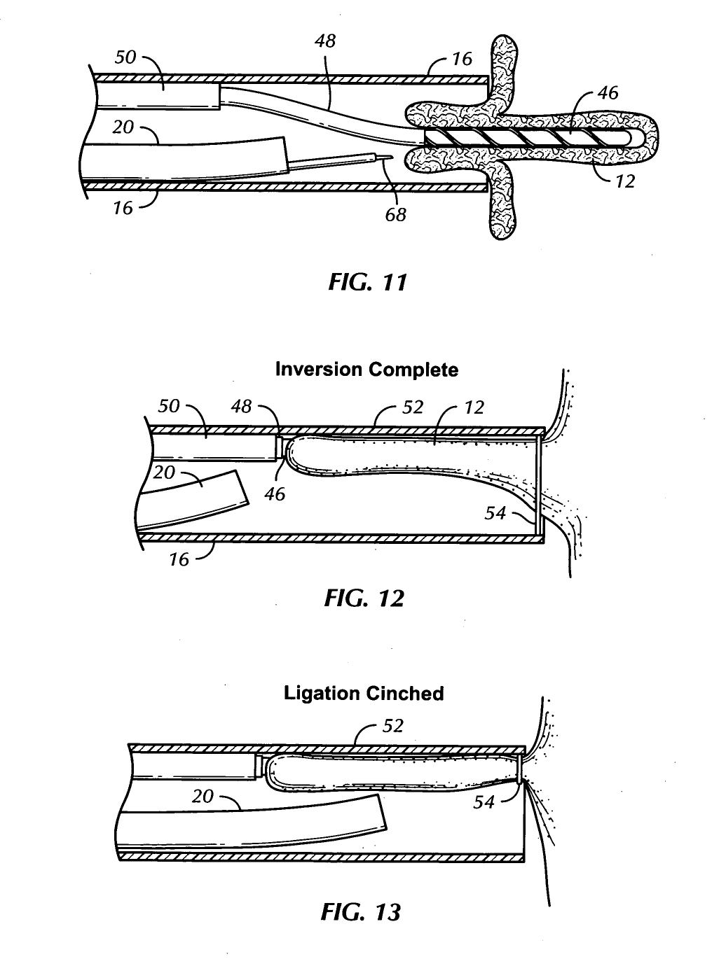

FIGS. 11-15 show how the above structure is used to invert tissue such as the appendix 12 for transanal removal of the tissue from the body. With the overtube 16 advanced through the anus to the appendix 12, the inversion catheter 46 is extended into the appendix 12 as shown in FIG. 11 by a surgeon manipulating the inverter control handle 64 shown in FIG. 10. The sealing sleeve 48 is also advanced to the opening of the appendix and may extend slightly into the appendix as shown for providing a vacuum seal. The above operations can be visualized using the endoscope 20 disclosed above and/or by using fluoroscopy or ultrasonic imaging as set forth previously. With the above-described manipulator withdrawn from the endoscope 20, a hollow cautery dissector catheter 68 can be advanced through the endoscope 20 for purposes to be shortly disclosed.

Once the inverter catheter 46 is positioned in the appendix, structure on the catheter 46 urges the appendix against the inverter catheter 46 so that upon proximal retraction of the inverter catheter 46 (by appropriate manipulation of the inverter control handle 64 shown in FIG. 10) the appendix inverts upon itself. Such structure is discussed further below, and can include vacuum holes that communicate with a vacuum lumen of the catheter 46 so that when the vacuum control knob 62 (FIG. 10) is manipulated to evacuate the lumen, the appendix is drawn against the catheter 46, with the sealing sleeve 48 functioning to prevent loss of vacuum within the appendix.

FIG. 12 illustrates inversion of the appendix 12 caused by retracting the inverter catheter 46. As shown, the appendix 12 is inverted through the ligating loop 54 into the overtube 16. Then, as illustrated in FIG. 13, the ligating loop 54 is tightly cinched around the appendix by appropriate pulling on the loop tensioning handle 58 shown in FIG. 10. If desired, the overtube 16 may be slightly retracted from the appendix at this point.

As understood herein, to facilitate inverting the appendix as additional tissue is being moved proximally, it may be necessary to use the cautery dissector catheter 68 prior to actually transecting the entire appendix to cauterize tissue during the inversion process to allow the appendix to fully invert.

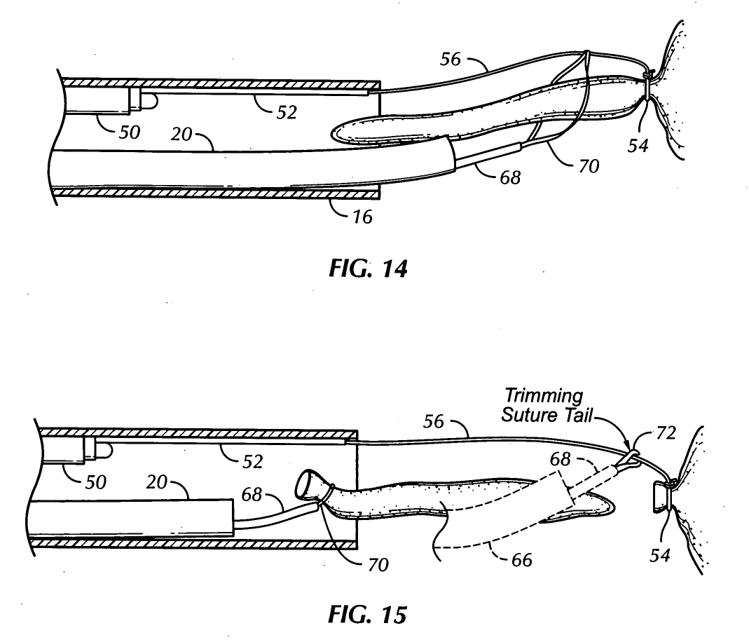

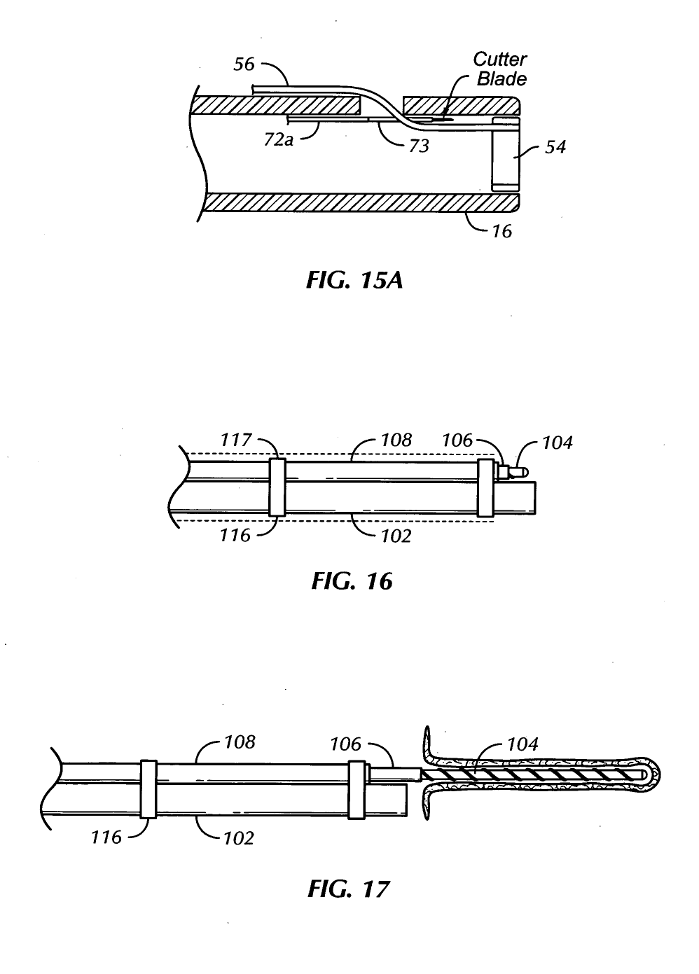

After fully inverting the appendix, as shown in FIG. 14, a cautery snare 70 may be extended from the cautery dissector catheter 68 (or equivalently another catheter that has been advanced through the endoscope after the dissector catheter 68 has been removed). Once the appendix is fully inverted, the snare 70 may be positioned around the inverted appendix and as shown in FIG. 15 tightened around the appendix and energized to transect the appendix. The dissector catheter 68 may then be retracted and the snare 70 used to grip the appendix to transanally retrieve the ligated appendix from the patient's body through the overtube 16. Or, the fully inverted and ligated appendix might not be transected; instead, it may be left in the patient to eventually slough off and pass through the bowels. The same process can be used with, e.g., the gall bladder, i.e., transanal inversion and ligation followed by leaving the organ in the body and allowing it to eventually slough off and pass through the bowels.

As shown in phantom in FIG. 15, the ligating cord 56 can be cut by a cutter 72 that can be extended through the cautery catheter 66. FIG. 15A shows that a cutter rod 72A may extend through the overtube 16. The rod 72A is formed with an aperture 73 having sharp inside edges, and the ligating cord 56 can pass through the aperture 73 so that when the rod 72A is moved proximally, the sharp inside edges of the aperture 73 cut the cord 56.

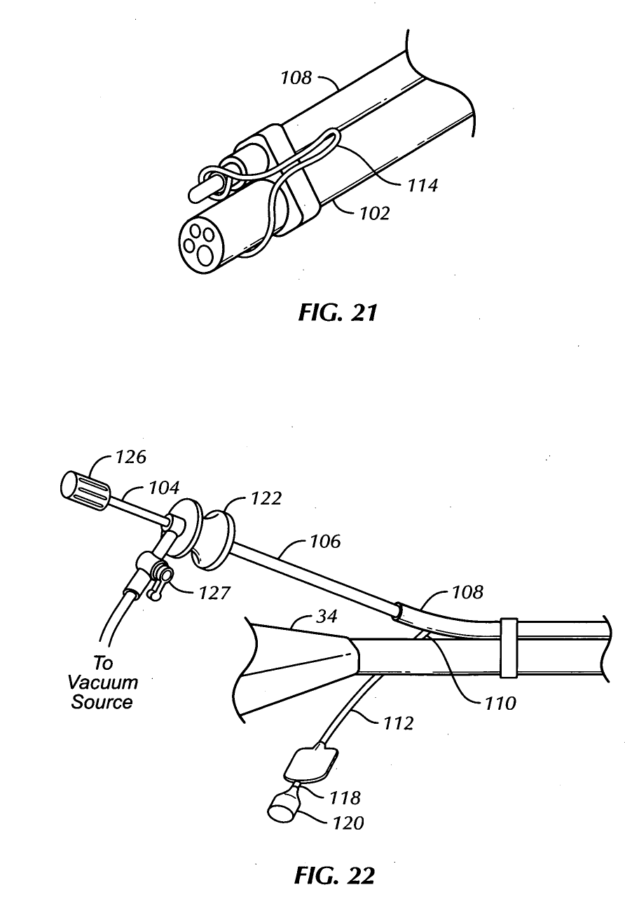

FIGS. 16-22 show an alternate catheter assembly 100 that in all essential respects may be identical to the catheter assembly 10 shown and discussed above, including having an endoscope 102, inverter catheter 104 with sealing sleeve 106 and outer cannula 108, and as best shown in FIGS. 19-21 inner and outer slidable hollow ligation tubes 110, 112 with ligating cord terminating in a ligation loop 114. The endoscope 102 may also contain pre-inversion manipulating devices as described above. However, instead of an overtube, the outer ligation tube 110, endoscope 102, and outer cannula 108 are held together in parallel by axially-spaced attachment clips or straps 116 that tightly surround the three components as shown. In some implementations a tubular sheath or condom 117 can encapsulate the assembly as shown to avoid trapping tissue between the clips 116.

With this structure, the catheter assembly 100 is advanced toward the appendix through the anus and the inversion catheter 104 is advanced into the appendix as shown in FIG. 17. It is to be understood that FIG. 19 is temporally out of sequence, in that in FIG. 19, shown to illustrate the ligating loop 114, the inversion catheter 104 is not yet advanced into the appendix.

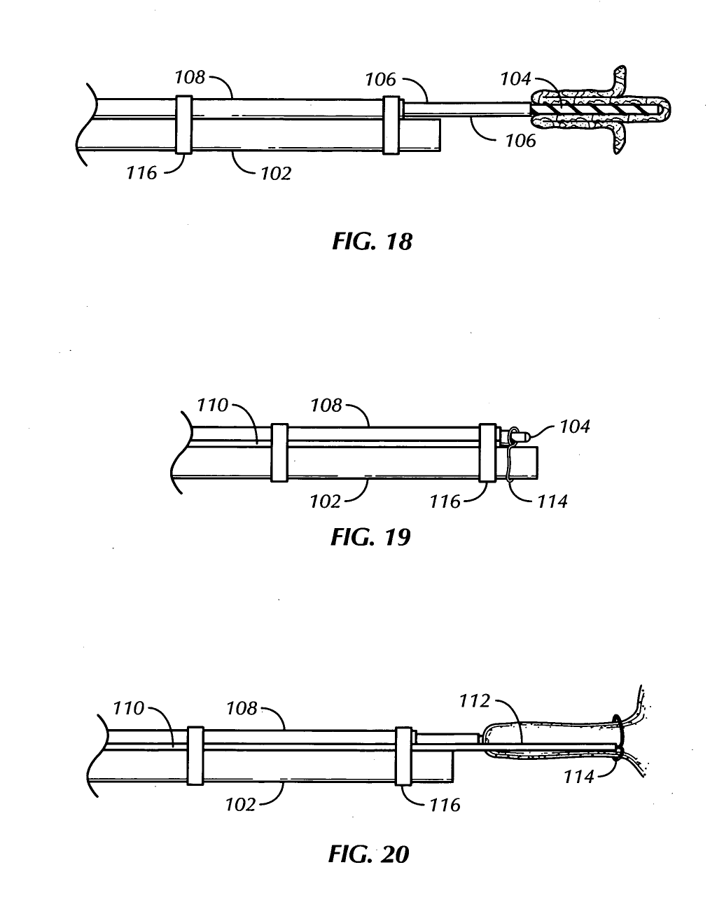

FIG. 18 shows the inversion catheter 104 advanced into the appendix and the appendix partially inverted and FIG. 20 shows the appendix fully inverted through the ligating loop 114, which has been advanced over the appendix by advancing the inner ligation tube 112 distally as shown. Appendix removal is then effected as disclosed above.

FIG. 22 shows non-limiting proximal controls for operating the catheter assembly 100 that are similar to the controls discussed previously. Specifically, as shown in FIG. 22, the inner ligation tube 112 extends proximally outside the patient, and a ligating cord 118 extends through the tube 112 and terminates in a loop tensioner handle 120. It will readily be appreciated that a surgeon can pull the handle 120 to tighten the loop 114.

Also, the inverter sealing sleeve 106 extends proximally beyond the patient, with the inverter catheter 104 extending beyond the sleeve 106 through a vacuum hub 122 and with the sealing sleeve 106 being coupled to the hub 122. The vacuum hub 122 is connected to a vacuum control knob 124 and to a source of vacuum for selectively evacuating a vacuum lumen in the inverter catheter 104 in accordance with disclosure below. The inverter catheter 104 terminates in a manipulable inverter control handle 126.

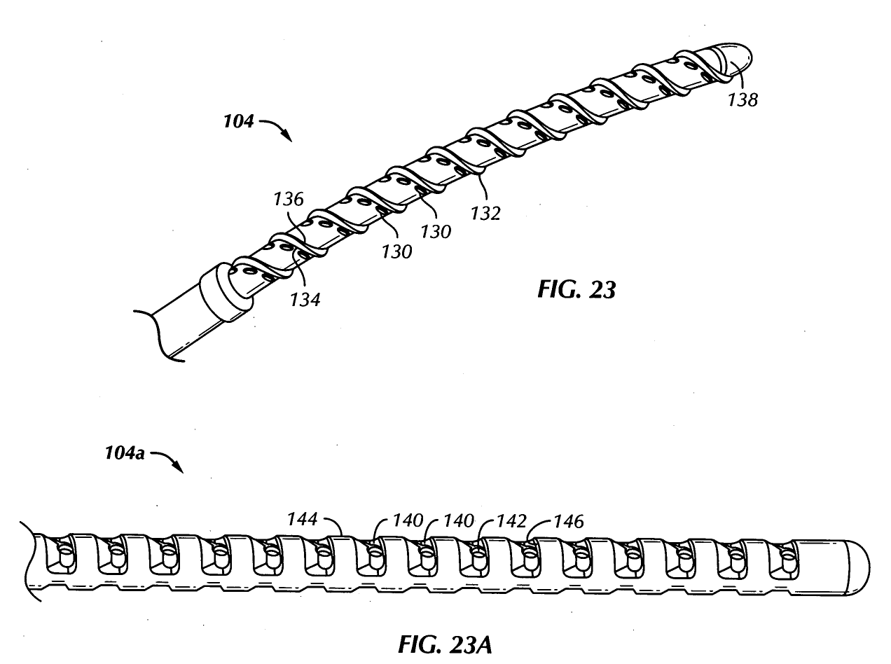

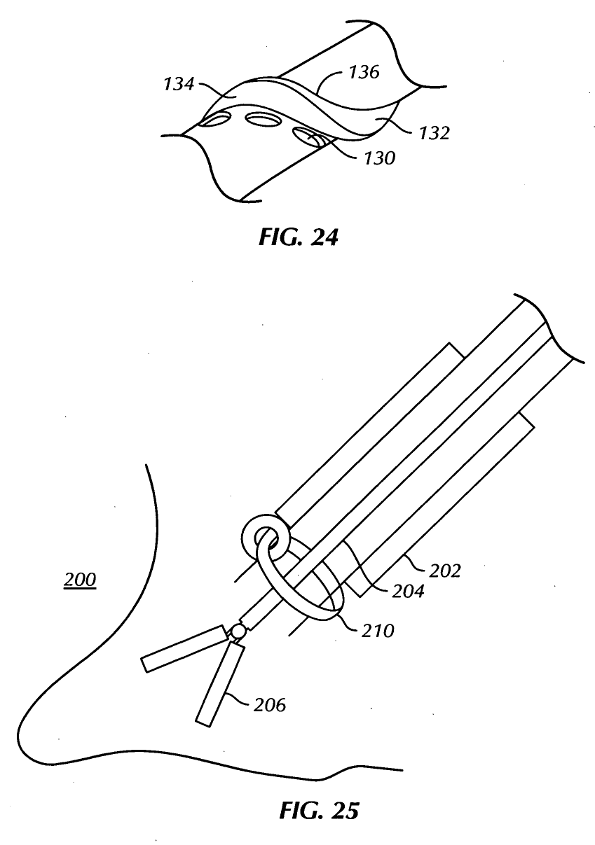

FIGS. 23 and 24 show non-limiting structure for urging the appendix against the inverter catheters discussed above. Taking the catheter 104 as an example, plural vacuum holes 130 are formed in the inverter catheter 104 and communicate, via a vacuum lumen of the inverter catheter 104, with a source of vacuum that is external to the patient. The external surface of the inverter catheter 104 can be formed with spiral ridges 132 as shown, with the vacuum holes 130 being formed closer to proximal sides 134 of the ridges 132 than to distal sides 136 of the ridges 132. The inverter catheter 104 may be provided with an atraumatic distal tip 138.

FIG. 23A shows an alternate inverting catheter 104A that has plural recesses 140. The recesses 140 are formed at least partially around the catheter 104A as shown parallel to each other and perpendicular to the axis of the catheter 104A. At least one vacuum hole 142 is formed in each recess 140 and communicates with the interior of the catheter 104A, with the vacuum holes 142 thus being recessed from the surface 144 of the catheter 104A. Preferably, the distal edges 146 of the recesses 140 are sharp, e.g., the catheter surface 144 is normal to the distal wall of a recess 140, to promote tissue adhesion to the evacuated catheter 104A.

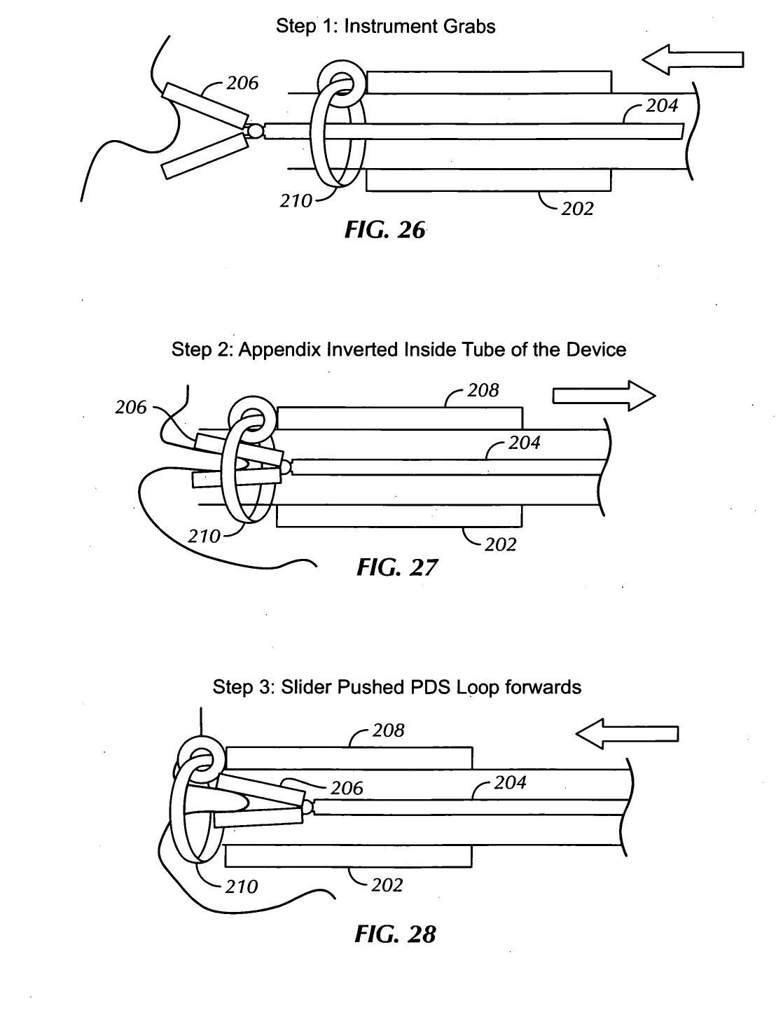

Now referring to FIGS. 25-30, an alternate transanal catheter is shown, generally designated 200, for drawing tissue such as a hemorrhoid, an appendix, or a gall bladder into an outer tube 202 for removing the tissue. In non-limiting implementations, a gripping slider 204 slides within the tube 202 and is biased to an open configuration, shown in FIGS. 25 and 26, wherein pivotably interconnected gripping arms 206 on the distal end of the slider 204 are spaced from each other, and a gripping configuration, shown in FIGS. 27 and 28, wherein the arms 206 are urged toward each other. In one implementation movement of the arms 206 is independent of the position of the arms 206 relative to the tube 202, i.e., the arms 206 can be moved by an operating device (not shown) outside the patient. Or, the arms 206 can be moved by virtue of the tube 202 being pushed distally relative to the slider 204 to ride up and over the arms 206. In either case, the arms 206 may be advanced out of the tube 202 to surround tissue as shown in FIG. 26 and then moved to grasp tissue and pull the tissue into the tube 202 as shown in FIGS. 27 and 28.

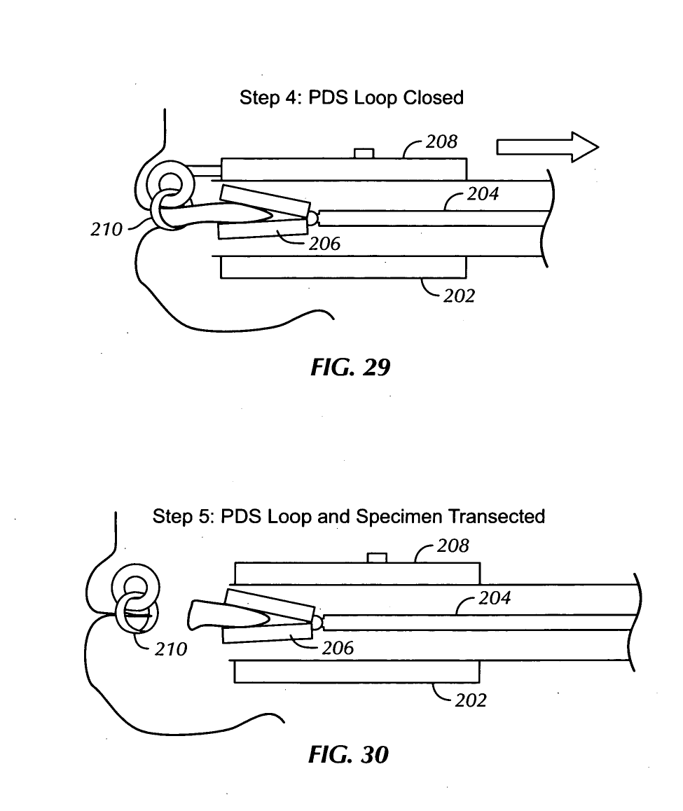

Once the tissue such as an appendix has been drawn into the tube 202, a ligating catheter 208, which may be supported by the tube 202, bears, at its distal end, a ligating loop 210 that surrounds the tissue to be transected. As shown in FIG. 29, the ligating cord that forms the loop 210 is retracted in accordance with principles set forth above to ligate the tissue, e.g., the appendix. As indicated in FIG. 30 any suitable transection device, including the cautery catheter 66 discussed above, may be used to remove the ligation loop and/or the tissue.

The catheter 200 shown in FIGS. 25-30 may include an endoscope in accordance with principles above, but in other implementations the catheter 200 is used without an endoscope. Instead, it is guided and visualized using fluoroscopy or ultrasonic imaging. When fluoroscopy is used, a radiopaque dye can be infused through the catheter into the tissue for imaging. Or, radiopaque markings may be provided on the catheter 200 in accordance with principles above. If it is desired to use ultrasonic imaging, the catheter 200 can include material that returns ultrasonic signals through the patient to a source of ultrasonic signals outside the patient in accordance with ultrasonic imaging principles known in the art, with the returned ultrasonic signals being used to generate an image of the catheter 200 and the tissue nearby the catheter 200.

Accordingly, with the above in mind:

In one embodiment the catheter 200 is designed for stage I & II hemorrhoids and is a hemorrhoid device. In one embodiment, the hemorrhoid device comprises a dedicated adaptor that draws the hemorrhoid inside a tube or instrument in order to stretch out the hemorrhoid to provide a larger target area. In one embodiment, the hemorrhoid device comprises a dedicated rapid closing loop to close and ligate the hemorrhoid. In one embodiment and as described further below, the hemorrhoid device comprises a textured loop to provide a better grip on the tissues. In one embodiment, the hemorrhoid device comprises an optional automatic transecting mechanism in the instrument so no other instrument needs to be introduced. This also simplifies the procedure for the user. In one embodiment, the hemorrhoid device comprises a vacuum to draw the hemorrhoid device into the tube. In one embodiment, the hemorrhoid device comprises a mechanical means for drawing the hemorrhoid tissue into the tube. In one embodiment, the hemorrhoid device comprises an automatic targeting that self centers the hemorrhoid within the tube. In one embodiment, the hemorrhoid device comprises a disposable instrument with a cartridge of loops. In one embodiment, the hemorrhoid device comprises a reusable instrument with a cartridge of ligation ties.

In one embodiment the catheter 200 is a natural orifice appendix system that grips the appendix from inside the caecum, and inverts the appendix and then ligates and transects the appendix from inside the caecum. In one embodiment, the natural orifice appendix system requires no incisions or coming out of the caecum because everything is done within the caecum. In one embodiment, the natural orifice appendix system comprises a tube system to draw the appendix inside the tube to control the appendix and to center the appendix within the tube. In one embodiment, the natural orifice appendix system uses an adjustable ligation tie, loop, suture, rapid closing loop, staples, or power to ligate the appendix and the vascular structures of the appendix from inside the caecum. In one embodiment, the natural orifice appendix system inverts the appendix and the vascular structures and applies a ligation transmurally. In one embodiment, the natural orifice appendix system comprises a mechanical means for drawing the appendix into the tube system. In one embodiment, the natural orifice appendix system comprises a vacuum means for drawing the appendix into the tube system. In one embodiment, the natural orifice appendix system comprises a flexible material to form the tube system so as to enter the body trans-anally and to be delivered along the length of the colon to the caecum and the appendix under any imaging means that includes but is not limited optical, ultrasound, CAT, and MRI. In one embodiment, the natural orifice appendix system comprises a self centering system that does not require direct imaging. In one embodiment, the natural orifice appendix system comprises a vacuum for clearing out the appendix prior to the ligation procedure. In one embodiment, the natural orifice appendix system is applicable colorectal or general surgery. In one embodiment, the natural orifice appendix system is designed to be minimally invasive such that no incision is required to remove an appendix.

In one embodiment, the natural orifice appendix system comprises an image guiding system that enables the natural orifice appendix system to be inserted transanally with a flexible GI scope. In one embodiment, the image guiding system comprises an internal or external imaging system such as but not limited to an Echo scan, CAT scan, or MRI scan.

In one embodiment, the natural orifice appendix system is delivered through the inner lumen of the colon to the point of the ileo-caecal junction and the appendix. In one embodiment, the natural orifice appendix system can also aspirate any puss from the area creating a clear operative field. In one embodiment, the natural orifice appendix system is configured to be a flexible tubular device of any diameter that fits into the colon. In one embodiment, the natural orifice appendix system comprises a terminal portion having a tube that is small enough to fit into the ileo-caecal junction. In one embodiment, the natural orifice appendix system comprises a reusable or disposable design or any combination of both.

In one embodiment, the natural orifice appendix system is configured to be guidable through any form of wires, pulleys, hydraulics, pneumatics, electronics, mechanical rods or any remote effectors mechanism.

In one embodiment, the natural orifice appendix system is configured to bend or rotate in any and all degrees of freedom to gain access to the structures.

In one embodiment, the natural orifice appendix system is configured to be controlled by a mechanism means that is either remote or integral to the system. In one embodiment, the natural orifice appendix system comprises an input device for guiding the system. In one embodiment, the natural orifice appendix system comprises a mechanical control input device. In one embodiment, the natural orifice appendix system comprises an electrical control input device. In one embodiment, the control input device is configured to be used by the operator externally to the patient.

In one embodiment, the natural orifice appendix system comprises a terminal hollow portion configured to receive the specimen to be inverted. In one embodiment, the natural orifice appendix system comprises a tubular structure configured to be a receptacle and a guide for advancing the adjustable ligation tie device or any other ligating mechanism. In one embodiment, the natural orifice appendix system comprises a terminal end configured to have any geometrical cross sectional shape, such as but not limited to cones, cylinders, and squares.

In one embodiment, the natural orifice appendix system comprises a grasping mechanism and/or a suction mechanism and/or any mechanism means for enabling the grasping of tissue such as but not limited to magnetic, ligations, sutures, needles or pins. In one embodiment, the natural orifice appendix system grasps and/or sucks the inside of the appendix to invert the appendix into the tubular housing as well as the terminal appendix artery.

In one embodiment, the natural orifice appendix system comprises an adjustable ligation tie that is configured to advance through the tubular housing to the appendix and vascular structures while the appendix is within the tubular housing and is under tension. In one embodiment, the natural orifice appendix system is configured to ligate the appendix with an adjustable ligation tie that is configured with the self locking system. In one embodiment, the natural orifice appendix system is configured to ligate the appendix with one or more adjustable ligation ties through the use of a single or multiple adjustable ligation tie delivery systems.

In one embodiment, the natural orifice appendix system is configured to provide tactile feedback to the operator through the design of the actuator.

In one embodiment, the natural orifice appendix system comprises an integral transection system that transects the specimen tissue. The integral transaction system may comprise any of the following: scissors, knives, blades, transecting loops, electrical, ultrasound, or hydrodissection. In one embodiment, the natural orifice appendix system is configured to remove the specimen and tail of the loop after integral transection system that transects the specimen tissue. In one embodiment, the natural orifice appendix system is configured to irrigate with solutions to wash and deliver antibiotics to the tissue area where the transecting occurred. In one embodiment, the natural orifice appendix system is configured with a suction means and irrigation port to allow the aspiration and irrigation of fluids.

In one embodiment, the natural orifice appendix system is configured with an adjustable ligation tie comprising an absorbable material. In one embodiment, the natural orifice appendix system is configured with an adjustable ligation tie comprising an non-absorbable materials.

In one embodiment, the natural orifice appendix system is configured to be removed from the body by being withdrawn through the colon.

The catheter 200 can be used in an incisionless, natural orifice procedure that is still image guided. It requires a lower learning curve and no abdominal entry. It does not enter the abdominal cavity and does not require the puncturing of any organs. This reduces infection, trauma and risk to patients.

The inverting of the structure into the device allows a unique way of accessing the external appendix without entering the abdomen.

The advantages of the natural orifice appendix system include but are not limited to: Incisionless/natural orifice; no organs or structures are punctured for access; image guided increases the safety profile—any method of imaging—direct or indirect; rapid access and delivery of the system with minimal anesthesia; simple and reliable ligation system; inverting the appendix so that the adjustable ligation tie remains inside the colon and does not invade the abdomen; inversion of the structures and ligating from within (not external ligation); secure ligation through the unique delivery system and below-described self locking clip system that can be both absorbable and non-absorbable; easy learning curve procedure that could be performed in the outpatient department; tissue grasping by mechanical or suction or use of magnets; can also be used on free structures without the need to invert them such as polyps etc.; use of suction/mechanical grasping/magnetic grasping to grasp the structure for the inversion; the natural orifice appendix system may also be applied and used in other GI surgery areas such as polyp removal, and full thickness tumor removal.

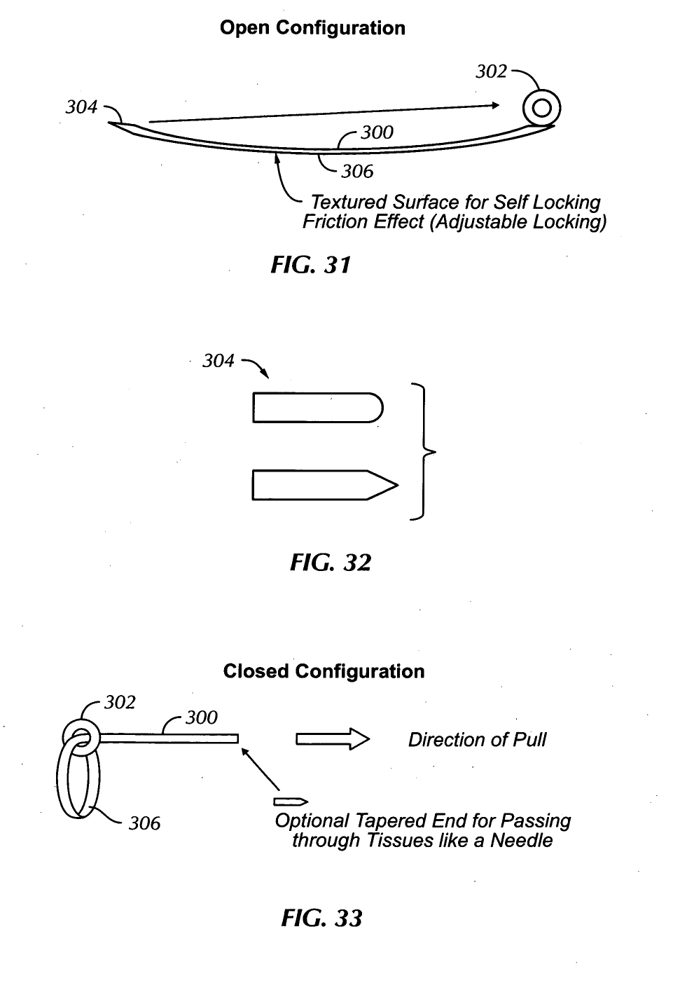

FIGS. 31-33 show non-limiting embodiments of the ligation loop 210 and associated cord shown in FIG. 29. As shown, an elongated absorbable or non-absorbable ligation body 300 can have a flat cross-section and thus is in the form of a long thin band, although round cross-sections may be used. A locking eye 302 is connected to one end of the body 300, and at the opposite end 304 the body 300 may terminate in a blunt or pointed configuration as shown in FIG. 32. The end 304 can be passed through the eye 302 to form the loop 210 discussed above. The side 306 of the body 300 that will form the inner surface of the loop 210 can have a textured surface to facilitate gripping tissue. The surface 306 may be textured by score lines, or small bumps or depressions, or other texturing structure. Owing to the texturing and the flatness of the body 300, improved tissue engagement is afforded.

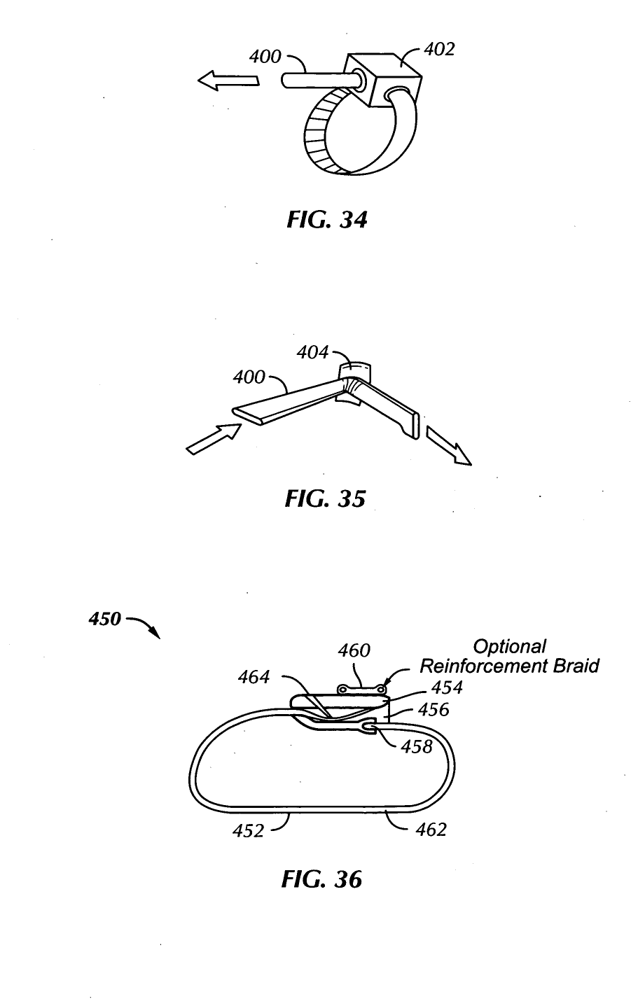

FIGS. 34 and 35 show that alternately, the loop 210 discussed above can be formed by drawing an elongated flat flexible ligation body 400 through a redirection box 402 that internally includes a redirection plate 404. As shown best in FIG. 35, the redirection plate 404 is configured to both rotate the body 400 ninety degrees about its long axis and to deflect the long axis of the body 400 ninety degrees, so that the body 400 extends away from the loop in a direction that is parallel to the axis of the loop. If desired, the ligation body 400 can have a textured surface 406 as shown in FIG. 34 to facilitate frictional self-locking between the redirection box 402 and the ligation body 400. A “differential” mechanism allows slippage of the body 400 vis-a-vis the redirection box 402 above a threshold pulling force. The part of the body 400 that extends from the loop can be pulled to tighten the loop.

Accordingly, with the above in mind, in one embodiment, the adjustable ligation tie comprises a cross sectional surface. In one embodiment, the adjustable ligation tie comprises a one piece flat starting configuration. In one embodiment, the adjustable ligation tie comprises a structure for passing the one piece flat configuration through to form a complete loop. In one embodiment, the adjustable ligation tie comprises a “textured” inner surface to increase tissue grip. In one embodiment, the adjustable ligation tie comprises a material that is absorbable. In one embodiment, the adjustable ligation tie comprises a material non-absorbable nature. In one embodiment, the adjustable ligation tie is designed to be hemostatic loop, using a broad surface area. In one embodiment, the adjustable ligation tie comprises a variable closing mechanism with defined steps. In one embodiment, the adjustable ligation tie comprises a ratcheted closure mechanism. In one embodiment, the adjustable ligation tie comprises a controlled closure mechanism.

In one embodiment, the adjustable ligation tie comprises a closure mechanism that creates a 360 degree enclosure that does not require a free pedicle to slip over. In one embodiment, the adjustable ligation tie comprises a semi-rigid configuration for better control over the device.

The above-described ligation tie is applicable to all fields of surgery where ligation is required. In confined spaces it is difficult to tie vascular structures or tissue structures with a standard suture because they are too flexible and require a “knot” to be formed, which often means a lack of consistency in ligating these structures which can lead to increased length of time or post operative bleeding complications. In contrast, the present ligation tie allows for a more rigid tie giving better control and fine consistent adjustments on closure, as well as reproducible ligation, especially in confined spaces. The above design features lead to greater tissue security.

The tie can be made of any absorbable or non-absorbable material and is formed as a single (or multiple component) device. It includes a strip of material that once threaded through the open eye of the device, closes the tie. This open eye allows complete adjustment of the closure of the tie (opening and closing). This system can be based upon a ratchet mechanism, or any type of infinitely adjustable friction mechanism.

The device can be made from any materials—metal, plastic, polymers of an absorbable or non-absorbable nature.

The inner surface of the tie can be textured by ridges, groves, dimples, raised or lowered sections. Any geometrical shape that adds frictional forces to the tissue to ensure that the adjustable ligation tie does not slide off the tissue may be employed.

The free end of the device can be a blunt configuration for when using on free pedicles or could be molded or formed into a “sharp” or “pointed” end that allows the free end to be passed through tissue if the structure to be ligated is not a free pedicle. The end of the device can be an integral portion of the tie or an additional structure that is in any way attached to the tie device.

The cross section of the device can vary in diameter, form and flexibility to meet the differing needs of the application. The thickness of the device can vary to accommodate any structure.

The semi-rigidity of the design allows for better control. The design ensures a 360 degree ligation with no milking. The knotless closing system allows for rapid closure of the device with ability to adjust the amount of closure in either direction. This adds a higher degree of control and accuracy.

The broader surface area stops any “cheese wire” effect. Further, the textured surface inside the tie allows for greater friction which reduces the possibility of tissue slippage. The cross sectional designs also reduce tissue slippage and tissue milking.

The integral “point” allows the device to be used as a tie around non-free pedicle structures allowing more structures to be ligated or transfixed.

The design allows for the tie to be secured at a distance such as during laparoscopic or endoscopic procedures without the need for manual (using the hands) adjustments. It also allows simple closure in any confined space where hand access is not feasible.

The elimination of knots or secondary clip mechanism to secure the closure is a unique advantage of the tie.

The system is self closing and locking and has semi-rigidity for control and security. A larger surface area is provided for reduced “cheese wire” effect. The textured inner tie surface provides for better friction on tissue, and the tie can be rapidly delivered and closed. The tie promotes adjustability of closing tension. The integrated/added sharp point provides for transfixation/ligation of non pedicles. The tie can be made from any material (polymer absorbable non absorbable metal), and there is no need for manual closure (using the hands directly) and no need for a secondary knot or clip to close/lock the device.

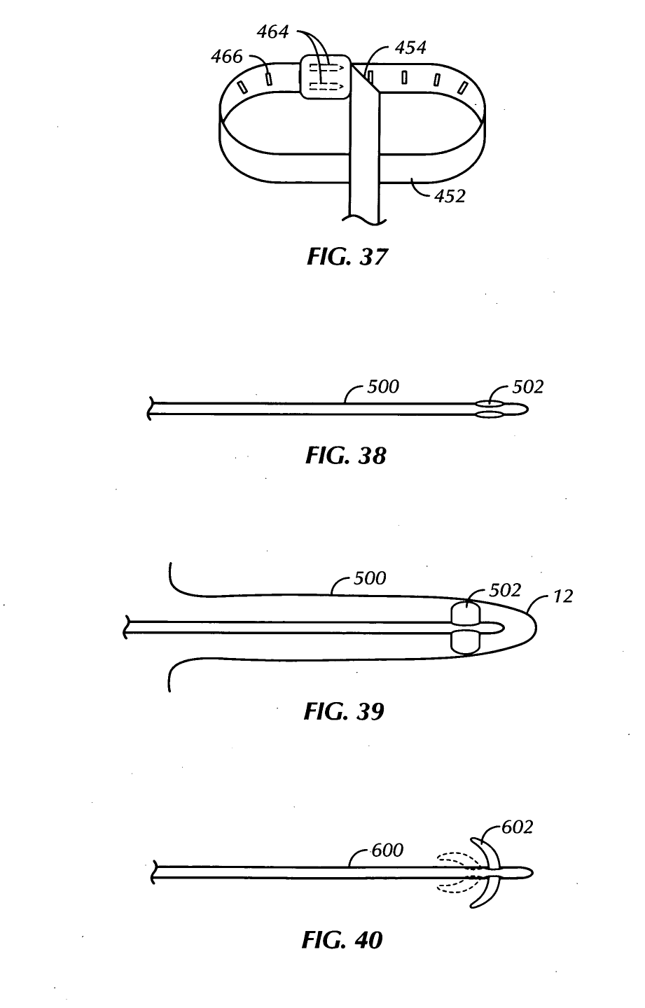

FIGS. 36 and 37 show an alternate ligating tie 450 that includes a flat flexible ligation body 452 that is folded over a bevel 454 establishing a forty five degree angle with respect to the axis of the body 452 at the point the body 452 passes through a slit 456 of the bevel 454. As shown, an end 458 of the body 452 is fastened to the bevel 454. The opposite (free) end 460 of the body 452, which may be reinforced, after passing through the bevel is turned ninety degrees as shown best in FIG. 37 relative to portions of the body 452 that have not yet been drawn through the bevel 454.

With this structure, the body 452 can be tensioned perpendicular to the plane of the loop 462 formed by the body 452. The body 452 may be made of a woven band such as is used for umbilical tape, and can have a width “W” of two to three millimeters. As the body 452 is tensioned, it is dragged over one or several one way spikes 464 that are formed on the bevel 454 or that are attached to the bevel 454. The spikes 464 are angled obliquely relatively to the body 452 and are oriented toward the direction taken by the body 452 as it is tightened, so that the body 452 may be pulled past the spikes 464 without engaging them but as soon as the tension on the body 452 is released, the body 452 locks onto the spikes.

In another implementation, the body 452 may be made of a plastic tape such as polypropylene ribbon which is tough yet pliable. The body 452 may have a matrix of braid embedded in it. With such a configuration, ratchet steps 466 (FIG. 37) may be formed on the body 452 and configured to slide past the spikes 464 when the body 452 is tensioned but to abut and thus engage the spikes 464 when the body 452 relaxes or indeed is attempted to be reversed in the bevel.

As mentioned above, the guidewire can be anchored in the appendix or other tissue once positioned inside. FIGS. 38 and 39 show a hollow guidewire 500 that has an inflatable balloon 502 at its distal tip, and the guidewire can be advanced into the appendix with the balloon deflated as shown in FIG. 38 and then the guidewire can be anchored into the appendix by inflating the balloon against the appendix walls as shown in FIG. 39. To remove the guidewire, the balloon is deflated. Saline or other suitable fluid may be used to inflate the balloon.

FIG. 40 shows that instead of a balloon, a guidewire 600 can have opposed elongated gently curved anchoring wings 602 that are pivotably engaged with the guidewire at its distal end between a widened anchoring configuration, shown in solid, and a narrowed travel position, shown in phantom. An actuator wire (not shown) may extend through the guidewire 600 and may be connected to the anchoring wings 602 to move them. To advance the guidewire 600 into the appendix, the wings 602 are moved to the travel position and the guidewire then advanced. Once placed in the appendix, the wings 602 are moved apart to the anchoring configuration to hold the guidewire in the appendix.

While the particular SYSTEMS AND METHODS FOR LESS INVASIVE RESOLUTION OF MALADIES OF TISSUE INCLUDING THE APPENDIX, GALL BLADDER, AND HEMORRHOIDS is herein shown and described in detail, it is to be understood that the subject matter which is encompassed by the present invention is limited only by the claims.

What is claimed is:

1. A tissue inversion catheter comprising:
an elongated catheter body transanally advanceable into a patient; and
an elongated inverter advanceable distally through the catheter body into a patient tissue sought to be inverted, the inverter including structure for urging the tissue against the inverter so that upon proximal retraction of the inverter the tissue inverts upon itself.

2. The inversion catheter of claim 1, wherein the structure for urging the tissue against the inverter includes plural vacuum holes formed in the inverter and communicating, via at least one vacuum lumen of the inverter, with a source of vacuum external to the patient.

3. The inversion catheter of claim 2, wherein the inverter defines an external surface formed with at least one of: spiral ridges, or recesses that are parallel to each other, the holes being formed in the ridges and/or the recesses.

4. The inversion catheter of claim 1, wherein the inverter reciprocates within a vacuum sealing sleeve in the catheter, the sleeve being positionable at the entry of the tissue being inverted while the inverter is disposed within the tissue to be inverted.

5. The inversion catheter of claim 1, wherein the structure for urging the tissue against the inverter includes a gripping element manipulable between a wide configuration, wherein the gripping element is positioned near the tissue, and a gripping configuration, wherein the gripping element grasps the tissue between gripping arms.

6. The inversion catheter of claim 1, comprising:
a ligator holder slidably disposed in the inversion catheter and having a distal end bearing a ligation element, the ligator holder being slidable past the tissue to a ligation point to slide the ligation element around the tissue; and
a tightener coupled to the ligator holder to cinch the ligation element around the tissue.

7. The inversion catheter of claim 6, comprising an excision tube and a snare advanceable distally away from the excision tube to snare the tissue after ligation for removing the tissue from the patient.

8. The inversion catheter of claim 1, wherein the catheter body is coupled to an endoscope to enable a person to view tissue as the catheter body is advanced into the patient.

9. The inversion catheter of claim 1, wherein the inversion catheter includes structure for facilitating ultrasonic and/or fluoroscopic guiding at least of the inverter.

10. The inversion catheter of claim 1, further comprising:
a manipulator extending through the inversion catheter and being advanceable into the tissue, the manipulator being coupled to an external manipulation control for moving the manipulator within the tissue to determine whether the tissue has restrictive anatomy or undesirable adhesions to nearby tissue prior to attempting inversion of the tissue.

11. The inversion catheter of claim 10, wherein the manipulator includes a stylet wire sufficiently rigid to straighten the tissue.

12. The inversion catheter of claim 10, wherein the manipulator includes a manipulator catheter having an actuator wire anchored near a distal end thereof, the manipulator catheter being sufficiently flexible to adhere to the contour of the tissue, the actuator wire being coupled to an actuator external to the patient and manipulable to cause the manipulator catheter to bend within the tissue, causing the tissue to thereby bend.

13. The inversion catheter of claim 6, wherein the ligation element includes a loop having a textured inner surface to facilitate gripping tissue.

14. The inversion catheter of claim 13, wherein the loop is formed by drawing an elongated flexible ligation member through a locking eye, the ligation member having a textured surface to facilitate frictional self-locking between the locking eye and the ligation member.

15. The inversion catheter of claim 6, wherein the ligation element includes a loop and a tightening member extending away from the loop a direction that is coaxial to the loop and that is manipulable to tighten the loop around tissue.

16. The inversion catheter of claim 1, comprising a guidewire extending through the catheter body and having at least one inflatable balloon at a distal end thereof to hold the guidewire within an appendix.

17. The inversion catheter of claim 1, comprising a guidewire extending through the catheter body and having at least one expandable wing at a distal end thereof to hold the guidewire within an appendix.

18. A method for appendectomy in a patient, comprising:
advancing an inverter into the appendix of the patient;
using the inverter to invert the appendix; and
removing the inverted appendix from the patient.

19. The method of claim 18, further comprising moving the appendix prior to inverting it to determine whether the appendix is undesirably adhered to nearby tissue.

20. The method of claim 18, further comprising ligating the inverted appendix.

21. The method of claim 18, comprising advancing an inverter into the appendix of the patient through the anus.

22. A method for appendectomy in a patient, comprising:
transanally advancing a catheter toward the appendix of the patient; and
using the catheter to remove the appendix from the patient.

23. The method of claim 22, comprising using the catheter to invert the appendix.

24. The method of claim 23, further comprising moving the appendix prior to inverting it to determine whether the appendix is undesirably adhered to nearby tissue.

25. The method of claim 24, further comprising ligating the inverted appendix.

26. A manipulator for moving an appendix, comprising:
a manipulator catheter advanceable into the appendix, the manipulator catheter being coupled to an external manipulation control for moving a manipulator element inside the appendix to determine whether the appendix has undesirable adhesions to nearby tissue.

27. The manipulator of claim 26, wherein the manipulator element includes a stylet wire sufficiently rigid to straighten the tissue.

28. The manipulator of claim 26, wherein an actuator wire is anchored near a distal end of the catheter, the manipulator catheter being sufficiently flexible to adhere to the natural contour of the appendix, the actuator wire being coupled to an actuator external to the patient and manipulable to cause the manipulator catheter to bend within the appendix, causing the appendix to thereby bend.

29. A method for determining whether an appendix is subject to undesirable adhesions to tissue nearby the appendix, comprising:
advancing a manipulator into the appendix;
moving the manipulator to thereby move the appendix; and
using fluoroscopy to determine whether the appendix has adhesions to nearby tissue.

30. A ligation element comprising:
a loop having a textured inner surface to facilitate gripping tissue.

31. The ligation element of claim 30, wherein the loop is formed by drawing an elongated flexible ligation member through a locking eye, the ligation member having a textured surface to facilitate frictional self-locking between the locking eye and the ligation member.

32. A ligation element comprising:
a loop defining a plane; and
a tightening member extending perpendicularly away from the plane of the loop and manipulable to tighten the loop around tissue.

33. A method for removing a gall bladder from a patient, comprising:
advancing an inverter into the gall bladder;
using the inverter to invert the gall bladder; and
removing the inverted gall bladder from the patient.

34. The method of claim 33, comprising advancing the inverter into the gall bladder of the patient through the anus.

35. A method for gall bladder removal in a patient, comprising: transanally advancing a catheter toward the gall bladder of the patient; and using the catheter to remove the gall bladder from the patient.
